(19) United States
(12) Patent Application Publication
(10) Pub. No.: US 2022/0082571 A1
(43) Pub. Date: Mar. 17, 2022

(54) METHODS FOR BINDING SITE IDENTIFICATION USING HYDROGEN EXCHANGE MASS SPECTROMETRY

(71) Applicant: Regeneron Pharmaceuticals, Inc., Tarrytown, NY (US)

(72) Inventors: Sisi Zhang, White Plains, NY (US); Hui Xiao, Ridgewood, NJ (US)

(21) Appl. No.: 17/472,605

(22) Filed: Sep. 11, 2021

Related U.S. Application Data
(60) Provisional application No. 63/077,220, filed on Sep. 11, 2020.

(51) Int. Cl. G01N 33/68; G01N 30/72

(52) U.S. Cl. CPC G01N 33/6848; G01N 30/7233; G01N 2333/948; G01N 2333/475; G01N 33/6857

(57) ABSTRACT

Methods for identifying a binding site between a protein pharmaceutical product and a host-cell protein (HCP) using hydrogen exchange mass spectrometry are provided. The present application also provides methods to modify protein pharmaceutical products to eliminate the cleavage or modification by HCPs. In addition, the present application provides methods to block the identified binding site in protein pharmaceutical products to eliminate the cleavage or modification by HCPs.

### CROSS-REFERENCE TO RELATED APPLICATION

This application claims priority to and the benefit of U.S. Provisional Patent Application No. 63/077,220, filed Sep. 11, 2020 which is herein incorporated by reference.

### FIELD

The present invention generally pertains to methods for identifying binding sites of host-cell proteins in proteins of interest or protein pharmaceutical products using hydrogen exchange mass spectrometry.

### BACKGROUND

The presence of residual host-cell proteins (HCPs) can cause potential safety risk for biopharmaceutical products and problems in manufacturing, especially in the presence of HCPs which have enzymatic activities. Since recombinant DNA technology has been used widely for producing biopharmaceutical products in host cells, it is necessary to remove impurities to obtain biopharmaceutical products having high purity. Any residual impurities after conducting the purification bioprocesses should be present at an acceptably low level prior to conducting clinical studies. In particular, residual HCPs derived from mammalian expression systems, for example, Chinese hamster ovary (CHO) cells, can compromise product safety, quality and stability. Sometimes, even trace amounts of particular HCPs can cause an immunogenic response or an undesirable modification. Thus, host cell proteins in drug products and during the manufacturing process need to be monitored.

Carboxypeptidase (G3H8V5), despite being commonly identified in drug substances, has never been reported to affect the integrity of monoclonal antibodies. Herein, carboxypeptidase is identified in a Fd′ drug substance and its effects on the stability of Fd′ drug are described. The amount of carboxypeptidase in Fd′ drug is also quantified, and findings suggest that the presence of carboxypeptidase as low as 10 ppm could compromise Fd′ drug stability. An extension of this study suggests that carboxypeptidase (G3H8V5) can also affect the stability of Fab protein, but not recombinant monoclonal antibodies engineered by IgG1 or IgG4.

Meanwhile, the interactions between HCPs and mAbs have not been fully understood. The most commonly accepted concept is that HCPs coelute with mAbs because of the non-specific binding between HCPs with mAbs. Herein, Hydrogen-deuterium Exchange Mass Spectrometry (HDX-MS) is applied in studying the interaction between Fd′ drug and carboxypeptidase and the specific binding site that allowed carboxypeptidase to cleave Fd′ drug is identified. The similar binding site is also found in Fab protein cleaved by Fabricator. However, no specific binding site is identified in Fd′ fusion drug and mAb drug.

It will be appreciated that a need exists for methods to identify and characterize HCP impurities that have enzymatic activities. In particular, these methods should be able to investigate the binding and/or enzymatic mechanisms of HCPs, such as identification of binding sites of HCPs in protein pharmaceutical products. These methods should provide robust, reliable and sensitive detection and characterization of enzymatic HCP impurities in biopharmaceutical products and in samples during manufacturing processes. Furthermore, these methods should be able to eliminate or block the enzymatic activities of HCPs based on protein pharmaceutical product modification.

### SUMMARY

Defining acceptable levels of HCP impurities has become a critical issue for using biological processing systems to manufacture biopharmaceutical products. There is a need to identify and characterize residual HCP impurities to investigate the binding and/or enzymatic mechanisms of HCPs. The present application provides methods to identify binding sites of HCPs in protein pharmaceutical products using hydrogen exchange mass spectrometry (HX-MS), for example, hydrogen/deuterium exchange mass spectrometry (HDX-MS). The present application also provides methods to modify protein pharmaceutical products to eliminate the cleavage or modification by enzymatic HCPs. In addition, the present application provides methods to block the identified binding site to eliminate the cleavage or modification by HCPs.

This disclosure provides a method for identifying a binding site between a protein of interest and a second protein. In some exemplary embodiments, the method comprises incubating a sample including the protein of interest and the second protein with deuterium oxide, adding a hydrolyzing agent to the sample to obtain a mixture with at least one digest, determining molecular weight data of the at least one digest in the mixture using a mass spectrometer, and correlating the molecular weight data of the at least one digest to data obtained from at least one known protein standard. In one aspect, the second protein is a host-cell protein. In one aspect, the sample is incubated with deuterium oxide for about 60 seconds to about 24 hours at room temperature. In another aspect, the method further comprises quenching the sample by adjusting the pH to about 2.3 and/or adjusting the temperature to about 0° C. In another aspect, the mixture is injected into a liquid chromatography system which is on-line with the mass spectrometer. In yet another aspect, the mass spectrometer is coupled to the liquid chromatography system.

In one aspect, the second protein is capable of cleaving the protein of interest. In another aspect, the cleavage involves an acidic residue, such as an aspartate residue or a glutamate residue. In another aspect, the cleavage occurs at the C-terminus of the protein of interest. In another aspect, the second protein is a carboxypeptidase. In one aspect, the second protein is a serine type carboxypeptidase. In yet another aspect, the protein of interest is a VEGF binding protein or a VEGF mini-trap. In a further aspect, the protein of interest is a Fab or F(ab′)2. In one aspect, the protein of interest is a protein pharmaceutical product, an antibody, a bispecific antibody, an antibody fragment, a Fab region of an antibody, an antibody-drug conjugate, a fusion protein, or a drug.

This disclosure, at least in part, provides a method for modifying a protein of interest to eliminate cleaving of a protein of interest by a second protein. In some exemplary embodiments, the method comprises (a) identifying a residue involved in a cleavage mechanism of the protein of interest by the second protein by: incubating a sample including the protein of interest and the second protein with deuterium oxide, adding a hydrolyzing agent to the sample to obtain a mixture with at least one digest, determining molecular weight data of the at least one digest in the mixture using a mass spectrometer, and correlating the molecular weight data of the at least one digest to data obtained from at least one known protein standard; and (b) mutating the identified residue to a second residue in order to eliminate cleaving of the protein of interest by the second protein.

In one aspect, the second protein is a host-cell protein. In one aspect, the sample is incubated with deuterium oxide for about 60 seconds to about 24 hours. In another aspect, the mixture is injected into a liquid chromatography system. In another aspect, the liquid chromatography system is on-line with the mass spectrometer. In yet another aspect, the mass spectrometer is coupled to the liquid chromatography system. In an aspect, the second protein is capable of cleaving the protein of interest. In another aspect, the cleavage involves an acidic residue, such as an aspartate residue or a glutamate residue. In another aspect, the cleavage occurs at the C-terminus of the protein of interest. In another aspect, the aspartate residue is mutated to a basic amino acid or a neutral amino acid. In yet another aspect, the glutamate residue is mutated to a basic amino acid or a neutral amino acid. In a further aspect, the second protein is a carboxypeptidase. In one aspect, the second protein is a serine type carboxypeptidase. In another aspect, the protein of interest is a VEGF binding protein or a VEGF mini-trap. In one aspect, the protein of interest is a Fab or F(ab′)2. In another aspect, the protein of interest is a protein pharmaceutical product, an antibody, a bispecific antibody, an antibody fragment, a Fab region of an antibody, an antibody-drug conjugate, a fusion protein, or a drug.

This disclosure, at least in part, provides a method for modifying a protein of interest to eliminate cleaving of the protein of interest by a second protein. In some exemplary embodiments, the method comprises (a) identifying a binding site between the protein of interest and the second protein by incubating a sample including the protein of interest and the second protein with deuterium oxide, adding a hydrolyzing agent to the sample to obtain a mixture having at least one digest, determining molecular weight data of the at least one digest using a mass spectrometer, and correlating the molecular weight data of the at least one digest to data obtained from at least one known protein standard; and (b) blocking the identified binding site in order to eliminate cleaving of the protein of interest by the second protein. In one aspect, the second protein is a host-cell protein. In one aspect, the sample is incubated with deuterium oxide for about 60 seconds to about 24 hours. In one aspect, the mixture is injected into a liquid chromatography system. In one aspect, the liquid chromatography system is on-line with the mass spectrometer. In one aspect, the mass spectrometer is coupled to the liquid chromatography system. In one aspect, the second protein is capable of cleaving the protein of interest. In one aspect, the cleavage involves an acidic residue, such as an aspartate residue or a glutamate residue. In another aspect, the cleavage occurs at the C-terminus of the protein of interest. In one aspect, the second protein is a carboxypeptidase. In one aspect, the second protein is a serine type carboxypeptidase. In one aspect, the protein of interest is a VEGF binding protein or a VEGF mini-trap. In one aspect, the protein of interest is a Fab or F(ab′)2. In one aspect, the protein of interest is a protein pharmaceutical product, an antibody, a bispecific antibody, an antibody fragment, a Fab region of an antibody, an antibody-drug conjugate, a fusion protein, or a drug. In one aspect, the method further comprises blocking the identified binding site using gene mutation, knockout, chemical modification, enzymatic modification, or combinations thereof.

These, and other, aspects of the present invention will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 shows the analysis of C-terminus deletion from VEGF mini-trap by HCP impurities at different intermediate time points including zero day (T0), one day (T1), four days (T4), or eight days (T8) using intact mass spectrometry according to an exemplary embodiment.

FIG. 2 shows the use of immunoprecipitation as a profiling method to identify HCPs according to an exemplary embodiment. Streptavidin-coated magnetic beads and biotinylated anti-HCP F550 antibodies were used to enrich HCPs according to an exemplary embodiment.

FIG. 3 shows the analysis of C-terminus deletion from F(ab′)2 fragment of MAB1 by HCP impurities at different intermediate time points using intact mass spectrometry according to an exemplary embodiment. The HCP impurities were present in VEGF mini-trap sample according to an exemplary embodiment.

FIG. 4 shows the analysis of C-terminus deletion from F(ab′)2 fragment of MAB1 by recombinant carboxypeptidase at different intermediate time points using intact mass spectrometry according to an exemplary embodiment.

FIG. 5 shows a three-dimensional structure of a protein complex containing VEGF mini-trap and carboxypeptidase based on the analysis results of HX-MS according to an exemplary embodiment. A strong protection region was identified in VEGF mini-trap, which indicated the binding site of carboxypeptidase according to an exemplary embodiment.

### DETAILED DESCRIPTION

During manufacturing of biopharmaceutical products, it is necessary to remove impurities to obtain biopharmaceutical products having high purities. The stability of drug formulations should be maintained during manufacturing, storage, shipment, handling and administration. Residual host cell proteins (HCPs) in biopharmaceutical products can present potential safety risks to patients due to compromising product stability and quality. In particular, some HCP impurities having enzymatic activities, such as proteases, can cause undesirable degradations, modifications or alterations of biopharmaceutical products. The identification and elimination of specific enzymatic HCPs are critical to ensuring product safety and stability. It is critical to understand the reaction mechanisms of enzymatic HCPs including working conditions of the enzymatic reactions, locations of enzymatic binding sites in the substrates, locations of cleavage/modification sites in the substrates, and the outcomes of enzymatic reactions, such as the chemical structures of the end products.

The present application provides methods to identify binding sites of HCPs in protein pharmaceutical products using HX-MS. The present application also provides methods to modify protein pharmaceutical products to eliminate the cleavage or modification of protein pharmaceutical products by enzymatic HCPs. The method of the present application can further include a step of mutating an identified residue in protein pharmaceutical products to eliminate the cleavage or modification by enzymatic HCPs, wherein the identified residue is involved in identified binding and/or enzymatic mechanisms. In addition, the present application can further include a step of blocking the identified binding site to eliminate the cleavage or modification of biopharmaceutical products by enzymatic HCPs.

In some embodiments, the present application provides a method to identify binding sites of a HCP in a protein pharmaceutical product using HX-MS. The method of the present application includes the steps of incubating a sample containing a protein pharmaceutical product and a HCP with deuterium oxide, adding a hydrolyzing agent to the sample to obtain a peptide mixture, determining molecular weight data of the peptide mixture using a mass spectrometer, and analyzing/correlating the molecular weight data of the peptide mixture with data of known protein standards. In one aspect, the peptide mixture is analyzed by a mass spectrometer which is coupled on-line with a liquid chromatography system. In another aspect, the present application provides a method for modifying a protein pharmaceutical product to eliminate cleavage of the protein pharmaceutical product by enzymatic HCPs. The method of modifying a protein pharmaceutical product includes the steps of identifying an amino acid residue which is involved in a cleavage mechanism of a HCP, and mutating the identified residue to a different residue in order to eliminate cleavage of the protein pharmaceutical product by the HCP. In one aspect, the method of modifying a protein pharmaceutical product includes the steps of identifying an amino acid residue which is involved in a binding site of a HCP, and blocking the identified binding site in order to eliminate cleavage of the protein pharmaceutical product by the HCP. The method of blocking the identified binding site includes gene mutation, knockout, chemical modification, enzymatic modification, or combinations thereof.

In one aspect, the HCP is a serine type carboxypeptidase which is capable of cleaving the protein pharmaceutical product at the C-terminus. In another aspect, the cleavage mechanism of the serine type carboxypeptidase involves an acidic residue, such as an aspartate residue or a glutamate residue. In yet another aspect, the protein pharmaceutical product is a VEGF binding protein, a VEGF mini-trap, a Fab, a F(ab′)2, an antibody, a bispecific antibody, an antibody fragment, an antibody-drug conjugate, a fusion protein, or a drug.

The demands of improving the quality, efficacy and safety of biopharmaceutical products have led to an increasing demand for identifying and characterizing HCP impurities which have enzymatic activities. This disclosure provides methods to satisfy the aforementioned demands. Exemplary embodiments disclosed herein satisfy the aforementioned demands by providing methods to identify a binding site between a protein pharmaceutical product and a HCP, to modify a protein pharmaceutical product to eliminate the cleavage by a HCP, and to block the identified binding site to eliminate the cleavage by a HCP.

The term “a” should be understood to mean “at least one”; and the terms “about” and “approximately” should be understood to permit standard variation as would be understood by those of ordinary skill in the art; and where ranges are provided, endpoints are included. As used herein, the terms “include,” “includes,” and “including,” are meant to be non-limiting and are understood to mean “comprise,” “comprises,” and “comprising,” respectively.

In some exemplary embodiments, the present application provides a method for identifying a binding site between a protein of interest and a second protein using hydrogen exchange mass spectrometry, the method comprising incubating a sample including the protein of interest and the second protein with deuterium oxide, adding a hydrolyzing agent to the sample to obtain a mixture with at least one digest, determining molecular weight data of the at least one digest in the mixture using a mass spectrometer, and correlating the molecular weight data of the at least one digest to data obtained from at least one known protein standard. In one aspect, the second protein is a host-cell protein. In another aspect, the protein of interest is a VEGF binding protein, a VEGF mini-trap, or a Fab or F(ab′)2. In a further aspect, the protein of interest is a protein pharmaceutical product, an antibody, a bispecific antibody, an antibody fragment, a Fab region of an antibody, an antibody-drug conjugate, a fusion protein, or a drug.

As used herein, the term “hydrogen exchange mass spectrometry”, “HX-MS”, “hydrogen/deuterium exchange mass spectrometry”, or “HDX-MS” refers to measurements of incorporation of deuterium into protein molecules using mass spectrometry (MS). HX-MS can be used to monitor structural and dynamic aspects of protein molecules in solution, since the exposure of a protein molecule to deuterium oxide (dideuterium oxide, D₂O, or heavy water) can induce rapid exchange from amide H (hydrogen) to amide D (deuterium) in disordered regions that lack stable hydrogen-bonding, e.g., conducting a hydrogen exchange reaction, a hydrogen/deuterium exchange reaction or a HDX reaction. Subsequently, mass spectrometry (MS)-based peptide mapping can be used to measure the mass shifts of individual protein segments. (Konermann et al., Hydrogen exchange mass spectrometry for studying protein structure and dynamics, Chem Soc Rev. March 2011, 40(3), page 1224-1234) After quenching the hydrogen exchange reaction, proteins are subjected to proteolysis to obtain a peptide mixture. Subsequently, the location and relative amount of deuterium exchanges in these peptides can be determined and analyzed based on mass shifts of individual peptides using MS. Deuterium contains a neutron as well as a proton. Since the deuterium nucleus is twice as heavy as the hydrogen nucleus, a protein molecule becomes heavier when its hydrogen is replaced by deuterium. Measurements of backbone amide hydrogen exchange rates (HID exchange rate) of protein molecules can provide detailed information regarding protein structure, dynamics, and interaction. Incorporation of amide deuterium into intact protein molecules can be measured by analyzing the derived peptide mixture using liquid chromatography coupled to an electrospray ionization source of MS. Within the native structures of protein molecules, hydrogen exchange rates can vary by several orders of magnitude. The hydrogen exchange rate is a function of solvent accessibility and hydrogen bonding. Amide hydrogens on the surface of protein molecules are bonded to water, which exhibit high hydrogen exchange rates. Amide hydrogens located within stable secondary structures of protein molecules exhibit low hydrogen exchange rates. (Johnson et al., Mass Spectrometric Measurement of Protein Amide Hydrogen Exchange of Apo- and Holo-Myoglobin”. Protein Science, 1994, 3 (12): 2411-2418)

As used herein, the term “protein” includes any amino acid polymer having covalently linked amide bonds. Proteins comprise one or more amino acid polymer chains, generally known in the art as “polypeptides.” “Polypeptides” refers to polymers composed of amino acid residues, related naturally occurring structural variants, and synthetic non-naturally occurring analogs thereof linked via peptide bonds related naturally occurring structural variants, and synthetic non naturally occurring analogs thereof “Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art. A protein may contain one or multiple polypeptides to form a single functioning biomolecule. A protein can include any of bio-therapeutic proteins, recombinant proteins used in research or therapy, trap proteins and other chimeric receptor Fc-fusion binding molecules, chimeric proteins, antibodies, monoclonal antibodies, polyclonal antibodies, human antibodies, and bispecific antibodies. In another exemplary aspect, a protein can include antibody fragments, nanobodies, recombinant antibody chimeras, cytokines, chemokines, peptide hormones, and the like. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a review discussing biotherapeutic proteins and their production, see Darius Ghaderi et al., Production platforms for biotherapeutic glycoproteins. Occurrence, impact, and challenges of non-human sialylation, 28 Biotechnology and Genetic Engineering Reviews 147-176 (2012). In some exemplary embodiments, proteins comprise modifications, adducts, and other covalently linked moieties. Those modifications, adducts and moieties include for example avidin, streptavidin, biotin, glycans (e.g., N-acetylgalactosamine, galactose, neuraminic acid, N-acetylglucosamine, fucose, mannose, and other monosaccharides), PEG, polyhistidine, FLAG tag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as, globular proteins and fibrous proteins; conjugated proteins, such as, nucleoproteins, glycoproteins, mucoproteins, chromoproteins, phosphoproteins, metalloproteins, and lipoproteins; and derived proteins, such as, primary derived proteins and secondary derived proteins.

As used herein, the term “hydrolyzing agent” refers to any one or combination of a large number of different agents that can perform digestion of a protein molecule. Non-limiting examples of hydrolyzing agents that can carry out enzymatic digestion include trypsin, endoproteinase Arg-C, endoproteinase Asp-N, endoproteinase Glu-C, outer membrane protease T (OmpT), immunoglobulin-degrading enzyme of Streptococcus pyogenes (IdeS), chymotrypsin, pepsin, thermolysin, papain, pronase, and protease from Aspergillus Saitoi. Non-limiting examples of hydrolyzing agents that can carry out non-enzymatic digestion include the use of high temperature, microwave, ultrasound, high pressure, infrared, and solvents (non-limiting examples are ethanol and acetonitrile). An example of non-enzymatic digestion also can include immobilized enzyme digestion (IMER), magnetic particle immobilized enzymes, and on-chip immobilized enzymes. For a review discussing the available techniques for protein digestion see Linda Switzar, Martin Giera & Wilfried M. A. Niessen, Protein Digestion: An Overview of the Available Techniques and Recent Developments, 12 Journal of Proteome Research 1067-1077 (2013). One or a combination of hydrolyzing agents can cleave peptide bonds in a protein or polypeptide, in a sequence-specific manner, generating a predictable collection of shorter peptides. Several approaches are available that can be used to digest a protein molecule. One of the widely accepted methods for digestion of protein molecules in a sample involves the use of proteases. Many proteases are available and each of them has their own characteristics in terms of specificity, efficiency, and optimum digestion conditions. Proteases refer to both endopeptidases and exopeptidases, as classified based on the ability of the protease to cleave at non-terminal or terminal amino acids within a peptide. Alternatively, proteases also refer to the six distinct classes—aspartic, glutamic, and metalloproteases, cysteine, serine, and threonine proteases, as classified by the mechanism of catalysis. The terms “protease” and “peptidase” are used interchangeably to refer to enzymes which hydrolyze peptide bonds. Proteases can also be classified into specific and non-specific proteases. As used herein, the term “specific protease” refers to a protease with an ability to cleave the peptide substrate at a specific amino acid side chain of a peptide. As used herein, the term “non-specific protease” refers to a protease with a reduced ability to cleave the peptide substrate at a specific amino acid side chain of a peptide. A cleavage preference may be determined based on the ratio of the number of a particular amino acid as the site of cleavage to the total number of cleaved amino acids in the protein sequences.

As used herein, the term “digest” refers to a derived product, such as a peptide, obtained from hydrolyzing one or more peptide bonds of a protein using a hydrolyzing agent. There are several approaches to carrying out hydrolysis or digestion of a protein in a sample using an appropriate hydrolyzing agent, for example, enzymatic digestion or non-enzymatic digestion.

As used herein, the term “host-cell protein” includes protein derived from the host cell and can be unrelated to the desired protein of interest. Host-cell protein can be a process-related impurity which can be derived from the manufacturing process and can include the three major categories: cell substrate-derived, cell culture-derived and downstream derived. Cell substrate-derived impurities include, but are not limited to, proteins derived from the host organism and nucleic acids (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

As used herein, “mini-trap” or “mini-trap binding molecule” refers to a molecule capable of binding to the same target as the fusion binding molecule which can be used to generate the mini-trap. Such mini-traps can include (i) chimeric polypeptides as well as (ii) multi-meric (e.g., dimeric) molecules comprising two or more polypeptides which are bound non-covalently or covalently, for example, by one or more disulfide bridges.

As used herein, “VEGF mini-trap” or “VEGF mini-trap binding molecule” can be capable of binding to vascular endothelial growth factor (VEGF) and are therapeutically useful for treating or preventing conditions and diseases which are treatable or preventable by inhibition of VEGF (e.g., VEGF₁₁₀, VEGF₁₂₁ or VEGF₁₆₅) such as angiogenic eye disorders and cancer. Such mini-traps include (i) chimeric polypeptides comprising one or more VEGF receptor domains as well as (ii) multimeric (e.g., dimeric) molecules comprising two or more polypeptides which are bound non-covalently, for example, by one or more disulfide bridges. Inhibition of VEGF includes, for example, antagonism of VEGF binding to VEGF receptor. The VEGF receptor domain components of the VEGF mini-traps of the present application include the immunoglobulin-like (Ig) domain 2 of VEGFR1 (Flt1) (R1D2), the Ig domain 3 of VEGFR2 (Flk1 or KDR) (Flk1D3) (R2D3), and/or the Ig domain 3 of VEGFR3 (Flk1 or KDR) (Flk1D3)(R3D3). Ig domains which are referenced herein, for example, R1D2, R2D3, R2D4 and R3D3, are intended to encompass not only the complete wild-type Ig domain, but also variants thereof which substantially retain the functional characteristics of the wild-type domain, for example, retain the ability to form a functioning VEGF binding domain when incorporated into a VEGF mini-trap. It will be readily apparent to one of skill in the art that numerous variants of the above Ig domains, which will retain substantially the same functional characteristics as the wild-type domain, can be obtained. In some aspects, the mini-trap binding molecule can include a variant of the mini-trap binding molecule. “Variant” or “binding molecule variant” as used herein can include a binding molecule that differs from a target binding molecule by virtue of at least one amino acid modification or a post-translational modification. The variant may refer to the binding molecule itself, a composition comprising the binding molecule, or the amino sequence that encodes it. Preferably, the binding molecule variant has at least one amino acid modification compared to the parent binding molecule, for example, from about one to about ten amino acid modifications, and preferably from about one to about five amino acid modifications compared to the parent. The binding molecule variant sequence herein will preferably possess at least about 80% homology with a parent binding molecule sequence, and more preferably at least about 90% homology, most preferably at least about 95% homology. In some aspects, the mini-trap binding molecule can be generated by digesting a fusion binding molecule.

As used herein, the term “Fab” or “F(ab′)2” refers to some specific antibody fragments, such as antibody fragments which are generated using proteases. Proteases suitable for generation of antibody fragments, such as a Fab or F(ab′)2 fragment, or VEGF mini-trap binding molecule, include endoproteinases, such as thrombin, papain, ficin, cysteine protease SpeB (FabULOUS) or cysteine protease IdeS (FabRICATOR®). Preferably the endoproteinase is the cysteine protease IdeS (FabRICATOR®). For example, IdeS specifically cleaves human IgG in the hinge region between the two glycine residues of the constant sequence ELLGGPS and SpeB cleaves in the hinge region between threonine and cysteine within the sequence KTHTCPPC. FabRICATOR® (Genovis #A0-FR1-020) is commercially available.

As used herein, “a protein pharmaceutical product” includes an active ingredient which can be fully or partially biological in nature. In one aspect, the protein pharmaceutical product can comprise a peptide, a protein, a fusion protein, an antibody, a monoclonal antibody, a bispecific antibody, an antigen, a vaccine, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, a fusion protein, an antibody, an antigen, a vaccine, a peptide-drug conjugate, an antibody-drug conjugate, a protein-drug conjugate, cells, tissues, or combinations thereof.

As used herein, an “antibody” is intended to refer to immunoglobulin molecules consisting of four polypeptide chains, two heavy (H) chains and two light (L) chains inter-connected by disulfide bonds. Each heavy chain has a heavy chain variable region (HCVR or VH) and a heavy chain constant region. The heavy chain constant region contains three domains, CH1, CH2 and CH3. Each light chain has a light chain variable region (VL) and a light chain constant region. The light chain constant region consists of one domain (CL). The VH and VL regions can be further subdivided into regions of hypervariability, termed complementarity determining regions (CDR), interspersed with regions that are more conserved, termed framework regions (FR). Each VH and VL can be composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” is inclusive of, but not limited to, those that are prepared, expressed, created or isolated by recombinant means, such as antibodies isolated from a host cell transfected to express the antibody. An IgG comprises a subset of antibodies.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, the phrase “bispecific antibody” includes an antibody capable of selectively binding two or more epitopes. Bispecific antibodies generally comprise two different heavy chains, with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain.

A typical bispecific antibody has two heavy chains each having three heavy chain CDRs, followed by a C_{H}1 domain, a hinge, a C_{H}2 domain, and a C_{H}3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats, such as, but not limited to triomab, knobs into holes IgG (kih IgG), crossMab, orth-Fab IgG, Dual-variable domains Ig (DVD-Ig), Two-in-one or dual action Fab (DAF), IgG-single-chain Fv (IgG-scFv), or κλ-bodies. The non-IgG-like different formats include Tandem scFvs, Diabody format, Single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 Journal of Hematology & Oncology 130; Dafne Müller & Roland E. Kontermann, Bispecific Antibodies, Handbook of Therapeutic Antibodies 265-310 (2014)).

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment contains sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

In one aspect, the sample of the present application is incubated with deuterium oxide for about 60 seconds to about 24 hours at room temperature. In one aspect, the method further comprises quenching the sample by adjusting the pH to about 2.3 and/or adjusting the temperature to about 0° C. In one aspect, the mixture is injected into a liquid chromatography system which is on-line with the mass spectrometer. In one aspect, the mass spectrometer is coupled to the liquid chromatography system. In another aspect, the mass spectrometer in the method of the present application is an electrospray ionization mass spectrometer, nano-electrospray ionization mass spectrometer, or a triple quadrupole mass spectrometer, wherein the mass spectrometer is coupled to a liquid chromatography system.

As used herein, a “mass spectrometer” includes a device capable of identifying specific molecular species and measuring their accurate masses. The term is meant to include any molecular detector into which a polypeptide or peptide may be eluted for detection and/or characterization. A mass spectrometer can include three major parts: the ion source, the mass analyzer, and the detector. The role of the ion source is to create gas phase ions. Analyte atoms, molecules, or clusters can be transferred into gas phase and ionized concurrently (as in electrospray ionization). The choice of ion source depends heavily on the application.

As used herein, the term “electrospray ionization” or “ESI” refers to the process of spray ionization in which either cations or anions in solution are transferred to the gas phase via formation and desolvation at atmospheric pressure of a stream of highly charged droplets that result from applying a potential difference between the tip of the electrospray needle containing the solution and a counter electrode. There are generally three major steps in the production of gas-phase ions from electrolyte ions in solution. These are: (a) production of charged droplets at the ES infusion tip; (b) shrinkage of charged droplets by solvent evaporation and repeated droplet disintegrations leading to small highly charged droplets capable of producing gas-phase ions; and (c) the mechanism by which gas-phase ions are produced from very small and highly charged droplets. Stages (a)-(c) generally occur in the atmospheric pressure region of the apparatus. In some exemplary embodiments, the electrospray ionization mass spectrometer can be a nano-electrospray ionization mass spectrometer.

As used herein, the term “triple quadrupole mass spectrometer” refers to a tandem mass spectrometer consisting of two quadrupole mass analyzers in series, with a (non-mass-resolving) radio frequency (RF)-only quadrupole between them to act as a cell for collision-induced dissociation. In a triple quadrupole mass spectrometer, a peptide sample is injected into a liquid chromatography system coupled with a MS instrument. The first quadrupole can be used as a mass filter to isolate peptides with a targeted m/z. The second quadrupole serves as a collision cell to break the peptide into fragments. The third quadrupole serves as a second mass filter for specified m/z fragments from the initial parent peptide. As used herein, the term “tandem mass spectrometry” includes a technique where structural information on sample molecules can be obtained by using multiple stages of mass selection and mass separation. A prerequisite is that the sample molecules can be transferred into gas phase and ionized intact and that they can be induced to fall apart in some predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on as long as one can obtain meaningful information or the fragment ion signal can be detectable. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time mass spectrometers ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited to, sequencing amino acids of the protein fragments, determining protein sequencing, determining protein de novo sequencing, locating post-translational modifications, or identifying post-translational modifications, or comparability analysis, or combinations thereof.

As used herein, the term “liquid chromatography system” or “chromatographic system” refers to a process in which a chemical mixture carried by a liquid or gas can be separated into components as a result of differential distribution of the chemical entities as they flow around or over a stationary liquid or solid phase. Non-limiting examples of chromatography include traditional reversed-phased (RP), ion exchange (IEX), mixed mode chromatography and normal phase chromatography (NP).

### EXEMPLARY EMBODIMENTS

Embodiments disclosed herein provide methods for identifying a binding site between a protein pharmaceutical product and a HCP using hydrogen exchange mass spectrometry to investigate the binding and/or enzymatic mechanisms of HCPs.

In some exemplary embodiments, the present application provides a method for identifying a binding site between a protein of interest and a second protein using hydrogen exchange mass spectrometry, the method comprising incubating a sample including the protein of interest and the second protein with deuterium oxide, adding a hydrolyzing agent to the sample to obtain a mixture with at least one digest, determining molecular weight data of the at least one digest in the mixture using a mass spectrometer, and correlating the molecular weight data of the at least one digest to data obtained from at least one known protein standard.

In one aspect, the hydrolyzing agent of the present application can perform enzymatic digestion including trypsin, endoproteinase Arg-C, endoproteinase Asp-N, endoproteinase Glu-C, outer membrane protease T (OmpT), immunoglobulin-degrading enzyme of Streptococcus pyogenes (IdeS), chymotrypsin, pepsin, thermolysin, papain, pronase, and protease from Aspergillus Saitoi.

In one aspect, the second protein of the present application is a HCP, an enzymatic HCP, a carboxypeptidase, a serine type carboxypeptidase, or a carboxypeptidase G3H8V5.

In one aspect, the protein of interest of the present application is a VEGF binding protein, a VEGF mini-trap, a Fab region of an antibody, a F(ab′)2 region of an antibody, a protein pharmaceutical product, an antibody, a bispecific antibody, an antibody fragment, an antibody-drug conjugate, a fusion protein, or a drug.

In some exemplary embodiments, the method of the present application comprises the step of incubating the sample with deuterium oxide for about 60 seconds to about 24 hours at room temperature and the step of quenching the sample by adjusting the pH to about 2.3 and/or adjusting the temperature to about 0° C.

In one aspect, the method of the present application comprises the step of incubating a sample containing the protein of interest and the second protein with deuterium oxide for about 60 seconds to about 24 hours, about 1 minute, about 2 minutes, about 3 minutes, about 5 minutes, about 8 minutes, about 10 minutes, about 15 minutes, about 20 minutes, about 30 minutes, about 40 minutes, about 50 minutes, about 55 minutes, about 1 hour, about 2 hours, about 3 hours, about 4 hours, about 5 hours, about 6 hours, about 8 hours, about 10 hours, about 12 hours, about 15 hours, about 18 hours, about 20 hours, about 22 hours, about 24 hours, about 36 hours, or about 72 hours. In one aspect, the method of the present application comprises the step of incubating a sample containing the protein of interest and the second protein with deuterium oxide at room temperature, at about 18° C., at about 25° C., at about 30° C., or at about 37° C. for about 60 seconds to about 24 hours, about 1 minute, about 2 minutes, about 3 minutes, about 5 minutes, about 8 minutes, about 10 minutes, about 15 minutes, about 20 minutes, about 30 minutes, about 40 minutes, about 50 minutes, about 55 minutes, about 1 hour, about 2 hours, about 3 hours, about 4 hours, about 5 hours, about 6 hours, about 8 hours, about 10 hours, about 12 hours, about 15 hours, about 18 hours, about 20 hours, about 22 hours, about 24 hours, about 36 hours, or about 72 hours.

In one aspect, the method of the present application comprises the step of quenching the sample by adjusting the pH of the reaction mixture to about 2.3, about 2.0, about 2.1, about 2.2, about 2.4, about 2.5, about 2.6, about 2.7, about 2.8, or about 2.9. In one aspect, the method of the present application comprises the step of quenching the sample by adjusting the temperature of the reaction mixture to about 0° C., about 1° C., about 2° C., about 3° C., about 4° C., or about 5° C.

It is understood that the method is not limited to any of the aforesaid HCP impurities, enzymatic binding site, hydrogen exchange mass spectrometry, VEGF binding protein, VEGF mini-trap, Fab fragment, F(ab′)2 fragment, protein pharmaceutical products, peptides, proteins, liquid chromatography system, or mass spectrometer.

The consecutive labeling of method steps as provided herein with numbers and/or letters is not meant to limit the method or any embodiments thereof to the particular indicated order. Various publications, including patents, patent applications, published patent applications, accession numbers, technical articles and scholarly articles are cited throughout the specification. Each of these cited references is incorporated herein by reference, in its entirety and for all purposes. Unless described otherwise, all technical and scientific terms used herein have the same meaning as commonly understood by one of ordinary skill in the art to which this invention belongs. This disclosure will be more fully understood by reference to the following Examples, which are provided to describe this disclosure in greater detail. They are intended to illustrate and should not be construed as limiting the scope of this disclosure.

### EXAMPLES

### Example 1. Identification and Characterization of Enzymatic HCPs

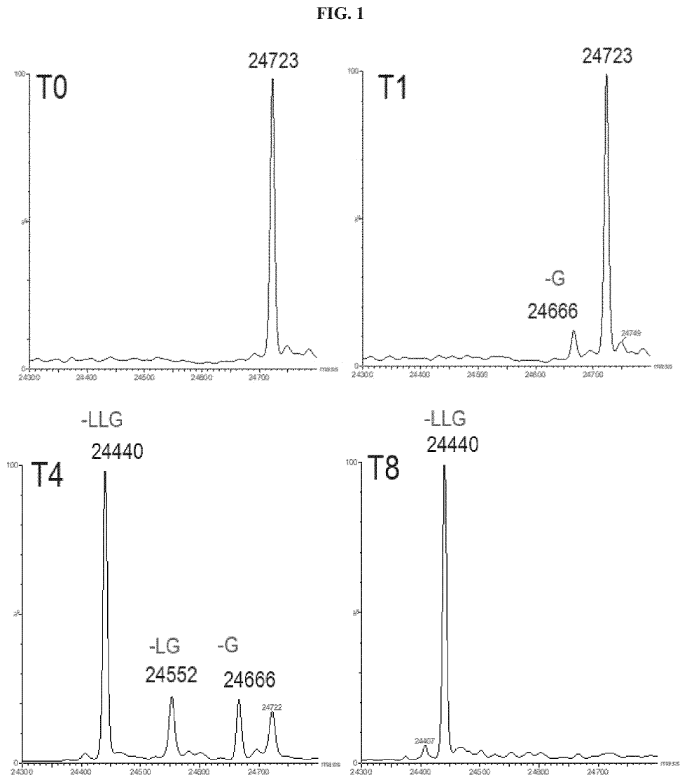

Samples containing VEGF mini-trap and HCP impurities were incubated at 37° C. for about 2 weeks or longer. Testing samples were collected at different intermediate time points, such as zero day (T0), one day (T1), four days (T4), or eight days (T8), and analyzed using intact mass spectrometry. FIG. 1 shows the analysis of C-terminus deletion from VEGF mini-trap at different intermediate time points using intact mass spectrometry. As shown in FIG. 1, the deletion of one glycine residue (G) from VEGF mini-trap was observed at time point T1. The deletions of glycine (G), leucine-glycine (LG) and leucine-leucine-glycine (LLG) were observed at time point T4. The deletions of leucine-leucine-glycine (LLG) were observed at time point T8. Data was collected until time point T16. No further cleavage was observed at time point T16. The results indicate the presence of stepped cleavage of C-terminus residues from VEGF mini-trap by enzymatic HCPs.

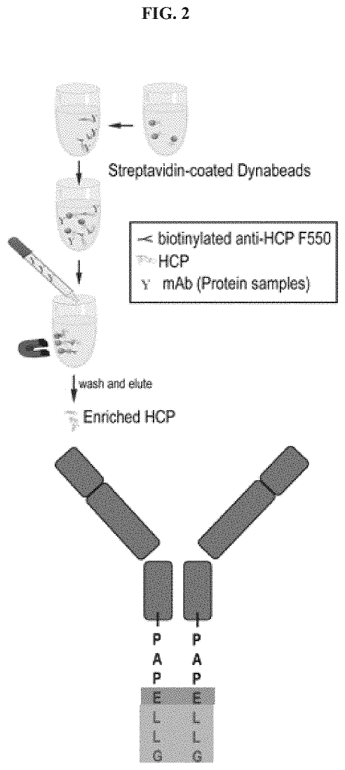

Immunoprecipitation was performed as a profiling method to identify HCPs that caused the stepped C-terminus deletion from VEGF mini-trap. Streptavidin-coated Dynabeads (magnetic beads) and biotinylated anti-HCP F550 antibodies were used to enrich HCPs. FIG. 2 shows the use of immunoprecipitation as a profiling method to identify HCPs. Carboxypeptidase G3H8V5 was identified for contributing to the stepped cleavage of C-terminus residues from VEGF mini-trap by immunoprecipitation and LC-MS/MS analysis. Specifically, 5 μL of 1 M acetic acid was added to 10 mg of protein sample and incubated at room temperature for 30 minutes. 110 μL of 10χ TBS and 20 μL of excess 1 M Tris-HCl (pH 8) were added to bring pH back to 7.5, then 25 μg of F550 biotinylated anti-HCP antibody was immediately added to each sample. Samples were incubated with gentle rocking at 4° C. overnight. 1.5 mg of magnetic beads were added to each sample after being washed and suspended in 1×TBS and incubated at room temperature with gentle rotating for 2 hours. Beads were then washed with HBS-T and 1×TBS, and eluted with 100 μL of 50% acetonitrile, 0.1 M acetic acid in MilliQ water by shaking at 800 rpm for 5 minutes twice. Each antibody sample was dried and resuspended in 20 μL of urea denaturing and reducing solution (8M urea, 10 mM DTT, 0.1 M Tris-HCl, pH 7.5), and incubated at 500 rpm at 56° C. for 30 minutes. 6 μL of 50 mM iodoacetamide was then added to each sample to mix and react at room temperature in the dark for 30 minutes. 50 μL of 20 ng/μL trypsin was added to each sample for digestion at 37° C., shaking at 750 rpm overnight. The digested samples were acidified with 4 μL 10% formic acid and 20 μL were transferred to glass vials for LC-MS/MS analysis. The rest were stored at −80° C.

Carboxypeptidase is a HCP which can be identified commonly in drug substances. Carboxypeptidase G3H8V5 is a serine type carboxypeptidase which can catalyze the hydrolysis of a peptide bond by clipping no more than three amino acid residues from the C-terminus of the protein substrate. The cleavage mechanism of carboxypeptidase G3H8V5 involves an acidic residue, such as an aspartate residue or a glutamate residue. The presence of carboxypeptidase G3H8V5 was further quantified using both immunoprecipitation enriched samples and unenriched samples as shown in Table 1. Peak areas of 3 peptides of G3H8V5 with highest abundance, including GAGHMVPTDKPR [m/z 633.3246²⁺], LFPEYK [m/z 398.7156²⁺] and LYQSMNSQYLK [m/z 687.8397²⁺], were used to compared with the peak areas of spiked-in hPLBD2 (a host protein, human putative phospholipase B-like 2) peptides GLEDSYEGR [m/z 1139.3698²⁺], QNLDPPVSR [m/z 1139.3690²⁺] and SVLLDAASGQLR [m/z 1448.4735²⁺] for quantitation of carboxypeptidase after IP enrichment, and indicated a concentration of 15 ppm. Unenriched samples were quantitated by direct digestion followed by PRM method using peak areas of 3 peptides of carboxypeptidase with highest abundance GAGHMVPTDKPR [m/z 633.3246²⁺], LFPEYK [m/z 398.7156²⁺] and LYQSMNSQYLK [m/z 687.8397²⁺] compared to peak areas of 3 peptides with highest abundance of VEGF mini-trap FLSTLTIDGVTR [m/z 661.8694²⁺], SDQGLYTCAASSGLMTK [m/z 895.4084²⁺] and SDTGRPFVEMYSEIPEIIHMTEGR [m/z 1397.6624²⁺], and indicated a concentration of 24.5 ppm.

TABLE 1
Quantification of carboxypeptidase G3H8V5.

| Carboxypeptidase G3H8V5 | VEGF mini-trap | hPLBD2 |
|---|---|---|
| IP Sample | | 15 ppm |
| Unenriched sample | 24.5 ppm | |

### Example 2. Confirmation of the Activity of Carboxypeptidase in VEGF Mini-Trap Sample

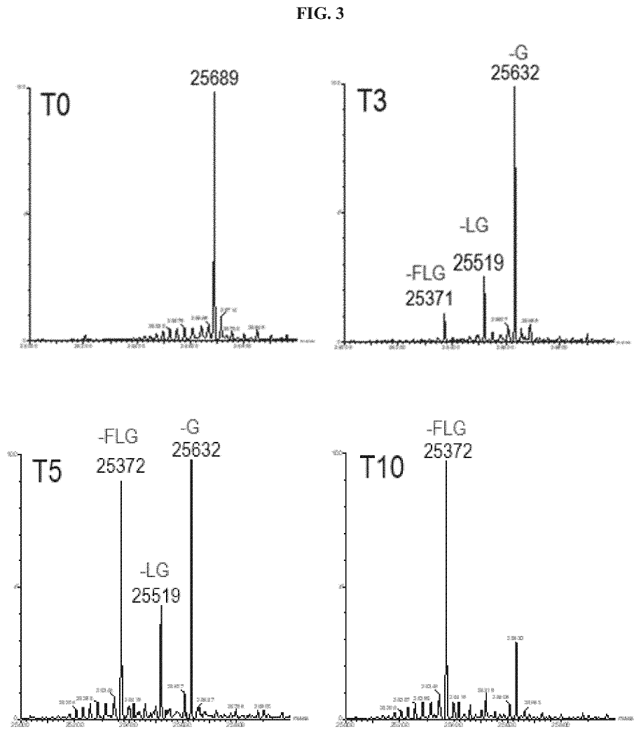

The activity of carboxypeptidase present as HCP impurities in VEGF mini-trap samples was further confirmed using a different protein substrate, a F(ab′)2 fragment of MAB1. Monoclonal antibody MAB1 was digested using FabRICATOR®, for example, cysteine protease IdeS to generate a specific F(ab′)2 fragment that shared similar C-terminus residues with VEGF mini-trap. Cysteine protease IdeS can digest antibodies at a specific site below the hinge to generate F(ab′)2 and Fc/2 fragments. This specific F(ab′)2 fragment contains a particular region at its C-terminus that can serve as a substrate for cleavage by carboxypeptidase. The VEGF mini-trap sample and the F(ab′)2 fragment of MAB1 were mixed together at a ratio of 1:1, wherein the VEGF mini-trap sample contained HCP impurities. The mixture was incubated at 37° C. for about 2 weeks or longer. Testing samples were collected at different intermediate time points, such as zero day (T0), three days (T3), five days (T5), or ten days (T10), and analyzed using intact mass spectrometry. FIG. 3 shows the analysis of C-terminus deletion from the F(ab′)2 fragment of MAB1 at different intermediate time points using intact mass spectrometry. As shown in FIG. 3, the deletions of glycine (G), leucine-glycine (LG) and phenylalanine-leucine-glycine (FLG) were observed at time points T3 and T5 at different proportions. The deletions of phenylalanine-leucine-glycine (FLG) were observed at time point T10. The results indicate the presence of stepped cleavage of C-terminus residues from the F(ab′)2 fragment of MAB1 by carboxypeptidase. The results indicate that the carboxypeptidase in HCP impurities clipped the C-terminuses of both VEGF mini-trap and F(ab′)2 fragment of MAB1, wherein the HCP impurities were present in the VEGF mini-trap sample.

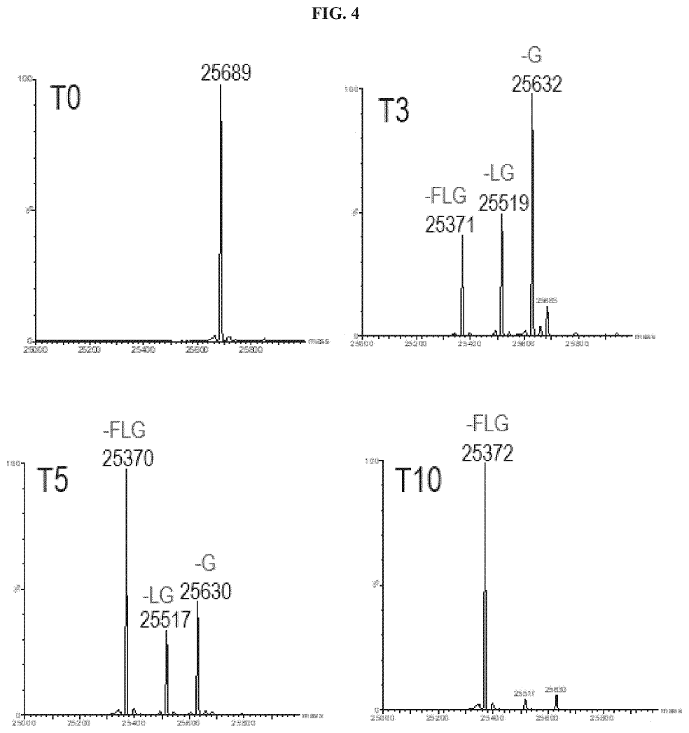

The F(ab′)2 fragment of MAB1 and recombinant carboxypeptidase were mixed together for further investigation of clipping mechanisms of the carboxypeptidase. The mixture was incubated at 37° C. for about 2 weeks or longer. Testing samples were collected at different intermediate time points, such as zero day (T0), three days (T3), five days (T5), or ten days (T10), and analyzed using intact mass spectrometry. FIG. 4 shows the analysis of C-terminus deletion from F(ab′)2 fragment of MAB1 by recombinant carboxypeptidase at different intermediate time points using intact mass spectrometry. As shown in FIG. 4, the deletions of glycine (G), leucine-glycine (LG) and phenylalanine-leucine-glycine (FLG) were observed at time points T3 and T5 at different proportions. The deletions of phenylalanine-leucine-glycine (FLG) were observed at time point T10. The results indicate the stepped cleavage of C-terminus residues from F(ab′)2 fragment of MAB1 by recombinant carboxypeptidase.

### Example 3. Binding Site Identification Using Hydrogen Exchange Mass Spectrometry (HX-MS)

Hydrogen exchange mass spectrometry (HX-MS) was used to identify the binding site of carboxypeptidase in VEGF mini-trap to understand the binding and clipping mechanisms of carboxypeptidase G3H8V5. The location and amino acid sequences of the binding site of carboxypeptidase G3H8V5 in VEGF mini-trap were further investigated. Since the exposure of a protein molecule to deuterium oxide can induce rapid exchange from amide H to amide D in disordered regions that lack stable hydrogen-bonding (Konermann et al.) or regions without protection, measurements of incorporation of deuterium into specific sites of protein molecules can be determined using mass spectrometry (MS) due to mass shifts.

The VEGF mini-trap sample including carboxypeptidase was prepared in solution with the addition of deuterium oxide for conducting a hydrogen exchange reaction, for example, a hydrogen/deuterium exchange reaction. The sample was incubated with deuterium oxide for about 60 seconds to about 24 hours at room temperature. Subsequently, the hydrogen exchange reaction was quenched by adjusting the pH to about 2.3 and/or adjusting the temperature to about 0° C. After quenching the hydrogen exchange reaction, the sample was subjected to proteolysis, such as trypsin digestion, to obtain a peptide mixture. The peptide mixture was injected into a liquid chromatography system which was coupled on-line with a mass spectrometer. The molecular weight data of the peptide mixture was analyzed and determined. Subsequently, the location and relative amount of deuterium exchanges in peptide mixture were determined and analyzed using MS based on mass shifts of peptides. Since the deuterium nucleus is twice as heavy as the hydrogen nucleus, a peptide molecule becomes heavier when its hydrogen is replaced by deuterium.

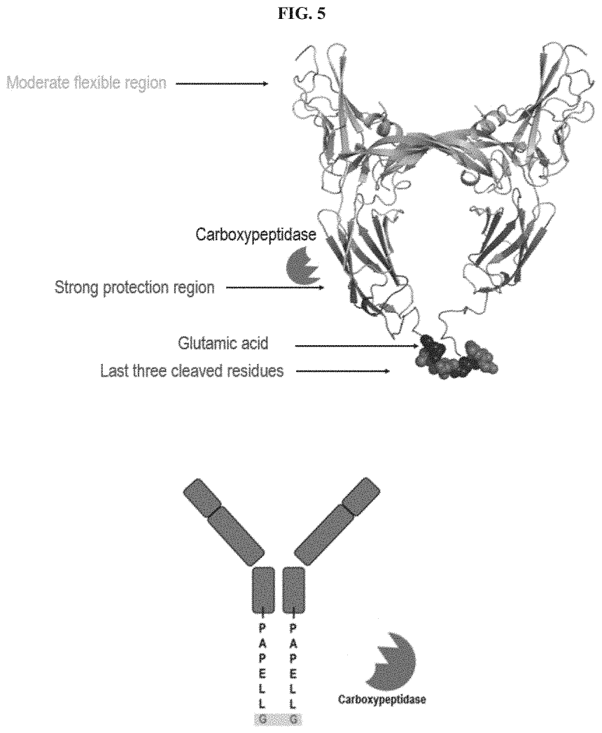

Measurements of backbone amide hydrogen exchange rates of the protein complex containing VEGF mini-trap and carboxypeptidase were conducted to investigate the structure, dynamics, and interaction of the protein complex. Within the structures of the protein complex, hydrogen exchange rates varied by several orders of magnitude, since the hydrogen exchange rate was a function of solvent accessibility and hydrogen bonding. Amide hydrogens on the surface of the protein complex exhibited high hydrogen exchanged rates. Amide hydrogens located in the binding site between two protein molecules within stable structures of the protein complex exhibited undetectable or low hydrogen exchange rates. When a region of a protein molecule was buried, the amide hydrogens in this region were protected from hydrogen exchanges. FIG. 5 shows a three-dimensional structure of a protein complex containing VEGF mini-trap and carboxypeptidase based on the analysis results of HX-MS. As shown in FIG. 5, a strong protection region was identified in VEGF mini-trap, which indicated the binding site of carboxypeptidase. The amino acids involved in the binding site were the amino acids at positions 170-186 of VEGF mini-trap, with the sequence of LTIDGVTRSDQGLYTCA. A glutamate residue in VEGF mini-trap was identified for its involvement in the clipping of three amino acid residues from the C-terminus of VEGF mini-trap by carboxypeptidase.

Successfully identifying the binding site and clipping mechanism of carboxypeptidase can lead to developing a method for eliminating the C-terminus cleavage of VEGF mini-trap. The specific glutamate residue involved with the cleavage mechanism can be mutated to a basic or neutral amino acid to eliminate the cleavage of VEGF mini-trap by carboxypeptidase. In addition, the identified binding site can be blocked to eliminate the cleavage of VEGF mini-trap by carboxypeptidase using gene mutation, knockout, chemical modification, enzymatic modification, or combinations thereof.

What is claimed is:

1. A method for identifying a binding site between a protein of interest and a second protein, the method comprising:
incubating a sample including the protein of interest and said second protein with deuterium oxide;
adding a hydrolyzing agent to the sample to obtain a mixture with at least one digest;
determining molecular weight data of said at least one digest in said mixture using a mass spectrometer; and
correlating said molecular weight data of said at least one digest to data obtained from at least one known protein standard.

2. The method of claim 1, wherein said second protein is a host-cell protein.

3. The method of claim 1, wherein said sample is incubated with deuterium oxide for about 60 seconds to about 24 hours.

4. The method of claim 3, wherein the sample is incubated at room temperature.

5. The method of claim 1 further comprising quenching said sample by adjusting the pH to about 2.3.

6. The method of claim 1 further comprising quenching the sample by adjusting the temperature to about 0° C.

7. The method of claim 1, wherein said mixture is injected into a liquid chromatography system.

8. The method of claim 7, wherein said liquid chromatography system is on-line with a mass spectrometer.

9. The method of claim 7, wherein a mass spectrometer is coupled to said liquid chromatography system.

10. The method of claim 1, wherein said second protein is capable of cleaving said protein of interest.

11. The method of claim 10, wherein said cleavage involves an acidic residue.

12. The method of claim 10, wherein said cleavage involves an aspartate residue or a glutamate residue.

13. The method of claim 1, wherein said second protein is a carboxypeptidase.

14. The method of claim 1, wherein said second protein is a serine type carboxypeptidase.

15. The method of claim 1, wherein said protein of interest is a VEGF binding protein or a VEGF mini-trap.

16. The method of claim 1, wherein said protein of interest is a Fab or F(ab′)2 generated using cysteine protease IdeS.

17. The method of claim 1, wherein said protein of interest is a protein pharmaceutical product, an antibody, a bispecific antibody, an antibody fragment, a Fab region of an antibody, an antibody-drug conjugate, a fusion protein, or a drug.

18. A method for modifying a protein of interest to eliminate cleaving of a protein of interest by a second protein, the method comprising:
identifying a residue involved in a cleavage mechanism of the protein of interest by a second protein by:
incubating a sample including said protein of interest and said second protein with deuterium oxide,
adding a hydrolyzing agent to said sample to obtain a mixture with at least one digest,
determining molecular weight data of said at least one digest in said mixture using a mass spectrometer, and
correlating said molecular weight data of said at least one digest to data obtained from at least one known protein standard; and
mutating the identified residue to a second residue in order to eliminate cleaving of the protein of interest by the second protein.

19. The method of claim 18, wherein said second protein is a host-cell protein.

20. The method of claim 18, wherein said sample is incubated with deuterium oxide for about 60 seconds to about 24 hours.

21. The method of claim 18, wherein said mixture is injected into a liquid chromatography system.

22. The method of claim 21, wherein said liquid chromatography system is on-line with a mass spectrometer.

23. The method of claim 21, wherein a mass spectrometer is coupled to said liquid chromatography system.

24. The method of claim 18, wherein said second protein is capable of cleaving said protein of interest.

25. The method of claim 18, wherein said cleavage involves an acidic residue.

26. The method of claim 18, wherein said cleavage involves an aspartate residue or a glutamate residue.

27. The method of claim 26, wherein said residue is aspartate and is mutated to a basic amino acid or a neutral amino acid.

28. The method of claim 26, wherein said residue is glutamate and is mutated to a basic amino acid or a neutral amino acid.

29. The method of claim 18, wherein said second protein is a carboxypeptidase.

30. The method of claim 18, wherein said second protein is a serine type carboxypeptidase.

31. The method of claim 18, wherein said protein of interest is a VEGF binding protein or a VEGF mini-trap.

32. The method of claim 18, wherein said protein of interest is a Fab or F(ab′)2 generated using cysteine protease IdeS.

33. The method of claim 18, wherein said protein of interest is a protein pharmaceutical product, an antibody, a bispecific antibody, an antibody fragment, a Fab region of an antibody, an antibody-drug conjugate, a fusion protein, or a drug.

34. A method for modifying a protein of interest to eliminate cleaving of a protein of interest by a second protein, comprising:
identifying a binding site between said protein of interest and said second protein by:
incubating a sample including said protein of interest and said second protein with deuterium oxide,
adding a hydrolyzing agent to said sample to obtain a mixture having at least one digest,
determining molecular weight data of said at least one digest using a mass spectrometer, and
correlating said molecular weight data of said at least one digest to data obtained from at least one known protein standard to identify a binding site; and
blocking said identified binding site in order to eliminate cleaving of said protein of interest by said second protein.

35. The method of claim 34, wherein said second protein is a host-cell protein.

36. The method of claim 34, wherein said sample is incubated with deuterium oxide for about 60 seconds to about 24 hours.

37. The method of claim 34, wherein said mixture is injected into a liquid chromatography system.

38. The method of claim 37, wherein said liquid chromatography system is on-line with a mass spectrometer.

39. The method of claim 37, wherein a mass spectrometer is coupled to said liquid chromatography system.

40. The method of claim 34, wherein said second protein is capable of cleaving said protein of interest.

41. The method of claim 34, wherein said cleavage involves an acidic residue.

42. The method of claim 34, wherein said cleavage involves an aspartate residue or a glutamate residue.

43. The method of claim 34, wherein said second protein is a carboxypeptidase.

44. The method of claim 34, wherein said second protein is a serine type carboxypeptidase.

45. The method of claim 34, wherein said protein of interest is a VEGF binding protein or a VEGF mini-trap.

46. The method of claim 34, wherein said protein of interest is a Fab or F(ab′)2 generated using cysteine protease IdeS.

47. The method of claim 34, wherein said protein of interest is a protein pharmaceutical product, an antibody, a bispecific antibody, an antibody fragment, a Fab region of an antibody, an antibody-drug conjugate, a fusion protein, or a drug.

48. The method of claim 34, further comprising blocking said identified binding site using gene mutation, knockout, chemical modification, enzymatic modification, or combinations thereof.
